(19) United States
(12) Patent Application Publication
(10) Pub. No.: US 2016/0100892 A1
(43) Pub. Date: Apr. 14, 2016

(54) Surgical Drape for Interventional Cardiology

(71) Applicant: William Wu, San Antonio, TX (US)

(72) Inventor: William Wu, San Antonio, TX (US)

(21) Appl. No.: 14/875,179

(22) Filed: Oct. 5, 2015

Related U.S. Application Data
(60) Provisional application No. 62/059,836, filed on Oct. 3, 2014.

(51) Int. Cl. A61B 19/08

(52) U.S. Cl. CPC A61B 19/088; A61B 2019/085; A61B 2019/086

(57) ABSTRACT

A surgical drape for interventional cardiology, the surgical drape comprising: a substantially rectangular impervious layer, wherein the substantially rectangular impervious layer is configured to be draped over a patient; one or more absorbent layers attached on one side of the substantially rectangular impervious layer, wherein the one or more absorbent layers cover substantially the substantially rectangular impervious layer; one or more pedal access regions through the substantially rectangular impervious layer and the one or more absorbent layers; one or more femoral access regions through the substantially rectangular impervious layer and the one or more absorbent layers; and one or more radial access regions through the substantially rectangular impervious layer and the one or more absorbent layers. Other embodiments are described and claimed.

### I. CROSS REFERENCE TO RELATED APPLICATIONS

This application claims the benefit of the filing date of U.S. Provisional Patent Application Ser. No. 62/059,836, filed on Oct. 3, 2014, entitled “Surgical Drape,” the entire disclosure of which is hereby incorporated by reference into the present disclosure.

### II. Background

The invention relates generally to surgical drapes. More particularly, the invention relates to an innovative surgical drape for interventional cardiology which comprises multiple access regions.

### III. Summary

In one respect, disclosed is a surgical drape for interventional cardiology, the surgical drape comprising: a substantially rectangular impervious layer, wherein the substantially rectangular impervious layer is configured to be draped over a patient; a first absorbent layer attached on one side of the substantially rectangular impervious layer, wherein the first absorbent layer covers substantially the length and partially the width of the substantially rectangular impervious layer; one or more pedal access regions through the substantially rectangular impervious layer and the first absorbent layer; and one or more femoral access regions through the substantially rectangular impervious layer and the first absorbent layer.

In another respect, disclosed is a surgical drape for interventional cardiology, the surgical drape comprising: a substantially rectangular impervious layer, wherein the substantially rectangular impervious layer is configured to be draped over a patient; a first absorbent layer attached on one side of the substantially rectangular impervious layer, wherein the first absorbent layer covers substantially the length and partially the width of the substantially rectangular impervious layer; one or more pedal access regions through the substantially rectangular impervious layer and the first absorbent layer; one or more femoral access regions through the substantially rectangular impervious layer and the first absorbent layer; a second absorbent layer attached on the same side of the substantially rectangular impervious layer as the first absorbent layer; a third absorbent layer attached on the same side of the substantially rectangular impervious layer as the first absorbent layer and the second absorbent layer; wherein the second absorbent layer and the third absorbent layer are adjacent to and on opposite the sides of the first absorbent layer; and one or more radial access regions through the substantially rectangular impervious layer and the second absorbent layer and/or the third absorbent layer.

In another respect, disclosed is a surgical drape for interventional cardiology, the surgical drape comprising: a substantially rectangular impervious layer, wherein the substantially rectangular impervious layer is configured to be draped over a patient; a first absorbent layer attached on one side of the substantially rectangular impervious layer, wherein the first absorbent layer covers substantially the length and partially the width of the substantially rectangular impervious layer; one or more pedal access regions through the substantially rectangular impervious layer and the first absorbent layer; one or more femoral access regions through the substantially rectangular impervious layer and the first absorbent layer; a patient side layer attached to the side of the substantially rectangular impervious layer opposite the side where the first absorbent layer is attached, wherein the one or more pedal access regions pass through the patient side layer and wherein the one or more femoral access regions pass through the patient side layer; a substantially circular adhesive ring attached to the side of the first absorbent layer opposite the side of the substantially rectangular impervious layer and substantially centered on one of the one or more femoral access regions; a plain liner adjacent to the side of the substantially rectangular impervious layer opposite the substantially circular adhesive ring, wherein the substantially circular adhesive ring is configured to removably hold the plain liner in place and wherein when the plain liner is removed from the surgical drape, the substantially circular adhesive ring is configured to adhere to the patient; double sided adhesive adjacent to the one or more pedal access regions, wherein the double sided adhesive is configured to adhere to the patient when the adhesive is exposed; a first stamp configured to indicate placement of the surgical drape relative to the patient, wherein the first stamp points in the direction of the head of the patient; and a second stamp configured to indicate placement of the surgical drape relative to the patient, wherein the second stamp points in the direction of the feet of the patient.

Numerous additional embodiments are also possible.

### IV. Brief Description of the Drawings

In Other objects and advantages of the invention may become apparent upon reading the detailed description and upon reference to the accompanying drawings.

FIG. 1 is a top plan view of a surgical drape with femoral and pedal access, in accordance with some embodiments.

FIG. 2 is a cross-sectional view of the surgical drape of FIG. 1 taken along the line A-A′, in accordance with some embodiments.

FIG. 3 is a cross-sectional view along the femoral access regions of a surgical drape with femoral and pedal access, in accordance with some embodiments.

FIG. 4 is a top plan view of a surgical drape with femoral, pedal, and radial access, in accordance with some embodiments.

FIG. 5 is a cross-sectional view of the surgical drape of FIG. 4 taken along the line B-B′, in accordance with some embodiments.

FIG. 6 is a cross-sectional view along the radial access regions of a surgical drape with femoral, pedal, and radial access, in accordance with some embodiments.

While the invention is subject to various modifications and alternative forms, specific embodiments thereof are shown by way of example in the drawings and the accompanying detailed description. It should be understood, however, that the drawings and detailed description are not intended to limit the invention to the particular embodiments. This disclosure is instead intended to cover all modifications, equivalents, and alternatives falling within the scope of the present invention as defined by the appended claims.

### V. Detailed Description

One or more embodiments of the invention are described below. It should be noted that these and any other embodiments are exemplary and are intended to be illustrative of the invention rather than limiting. While the invention is widely applicable to different types of systems, it is impossible to include all of the possible embodiments and contexts of the invention in this disclosure. Upon reading this disclosure, many alternative embodiments of the present invention will be apparent to persons of ordinary skill in the art.

Surgical drapes currently used in interventional cardiology provide for radial access and/or femoral access. Depending on the circumstances, pedal access for the procedure is sometimes desired by the surgeon. A need exists, for efficiency and flexibility, for a surgical drape, especially for interventional cardiology procedures, for a surgical drape comprising femoral access and pedal access and in some embodiments, radial, femoral, and pedal access. The invention disclosed in the subsequent description and figures provide for just such surgical drapes.

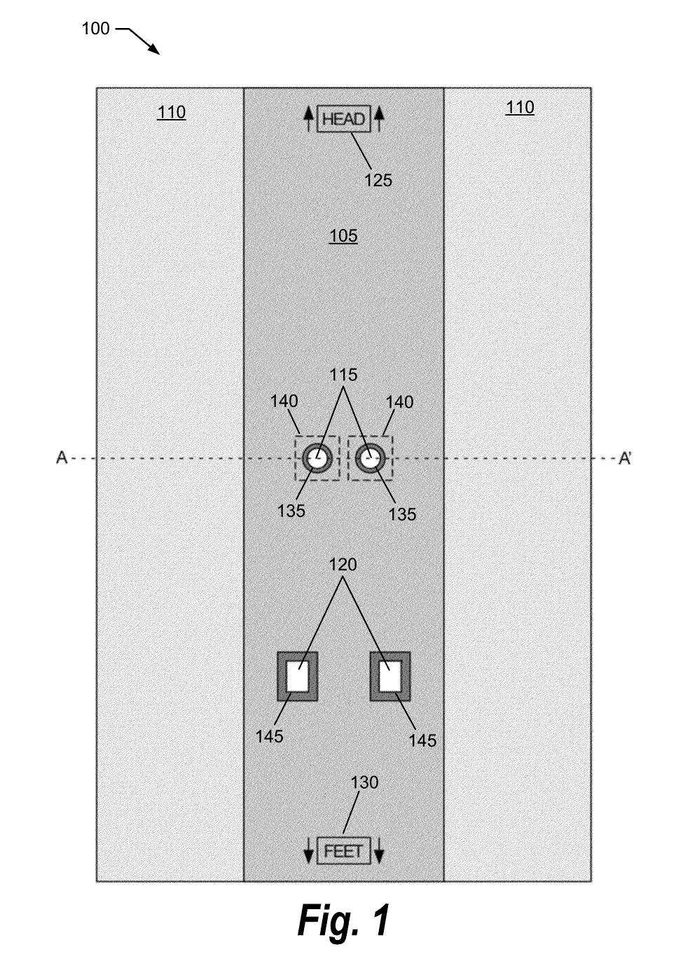

FIG. 1 is a top plan view of a surgical drape with femoral and pedal access, in accordance with some embodiments.

In some embodiments, the surgical drape 100 comprises a substantially full length absorbent layer 105 attached to a substantially rectangular, full length, impervious layer 110 with femoral access regions 115 and pedal access regions 120. In some embodiments, a head stamp 125 is indicated on the absorbent layer 105 along the edge of the absorbent layer 105 opposite the pedal access regions 120 across the femoral access regions 115 and a foot stamp 130 is indicated on the absorbent layer 105 along the edge of the absorbent layer 105 on the end of the pedal access regions 120. The femoral access regions 115 and pedal access regions 120 extend through both the absorbent layer 105 and the impervious layer 110. The absorbent layer 105 is designed to manage the fluids that exist during a surgical procedure and the impervious layer 110 reduces the movement of these fluids to unwanted areas.

In some embodiments, the femoral access regions 115 comprise two substantially circular fenestrations into the absorbent layer 105 which permit access to the groin area of a patient. In some embodiments, the femoral access regions 115 comprise circular fenestrations of approximately 5 inches. On the patient side of the absorbent layer 105, an adhesive 135 surrounds each of the femoral access regions 115. In the new, sterile condition, the femoral access regions 115 each have plain liners 140 attached to the patient side of the surgical drape by the adhesive 135. Since the plain liners 140 are attached on the patient side of the drape, their position is shown as dashed lines, indicating that part of the plain liners 140 are hidden from view when the drape is viewed in a top plan view. When the surgical drape is to be used, the appropriate plain liner 140 is removed in order to expose the adhesive 135 so that the surgical drape may be adhered to the patient at the location of the patient's body where access to the patient is desired.

In some embodiments, the pedal access regions 120 comprise two substantially rectangular fenestrations into the absorbent layer 105 which permit access to the ankle/foot area of the patient and a double sided adhesive 145 surrounds each of the pedal access regions 120. In some embodiments, the pedal access regions 120 comprise substantially rectangular fenestrations of approximately 4 inches by 5 inches. When the surgical drape is to be used, the appropriate double sided adhesive 145 is exposed so that the surgical drape may be adhered to the patient at the location of the patient's body where access to the patient is desired.

In some embodiments, a third layer is attached to the impervious layer 110 on the patient side opposite the absorbent layer 105. In such an embodiment, the femoral access regions 115 and pedal access regions 120 also extend through the third layer. The third layer is used to provide for a comfortable material to be in contact with the patient. The third layer may comprise a woven or nonwoven fabric layer and/or a layer of fibers.

In some embodiments, the absorbent layer 105 comprises a woven or nonwoven fabric layer and/or a layer of fibers. The fabric/fibers may comprise a polymeric film-forming material, such as polypropylene, an absorbent material of Airtex material, Angio material, and/or SMS (“Spunbond+Meltblown+Spunbond Nonwovens”) fabric. The absorbent layer has an average layer thickness of less than about 10 mils.

In some embodiments, the impervious layer 110 comprises a polymeric film-forming material, such as polyethylene, and has an approximate length of 135 inches, an approximate width of 84 inches, and an average layer thickness of less than about 2 mils.

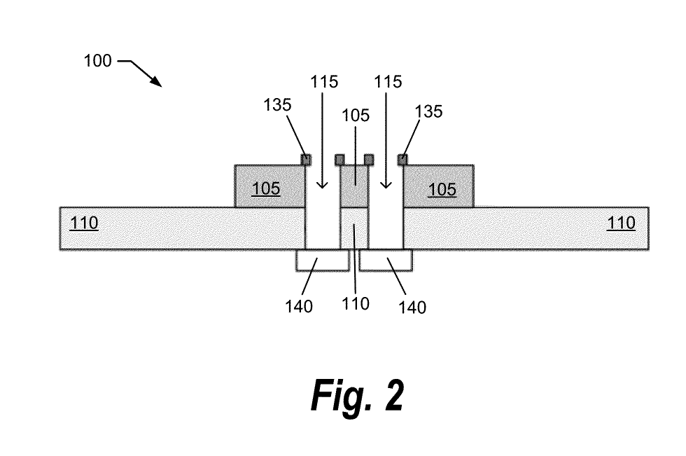

FIG. 2 is a cross-sectional view of the surgical drape of FIG. 1 taken along the line A-A′, in accordance with some embodiments.

In some embodiments, the surgical drape 100 comprises a substantially full length absorbent layer 105 attached to a substantially rectangular, full length, impervious layer 110 with femoral access regions 115 and pedal access regions. The femoral access regions 115 and pedal access regions extend through both the absorbent layer 105 and the impervious layer 110. The absorbent layer 105 is designed to manage the fluids that exist during a surgical procedure and the impervious layer 110 reduces the movement of these fluids to unwanted areas.

In some embodiments, the femoral access regions 115 comprise two substantially circular fenestrations into the absorbent layer 105 which permit access to the groin area of a patient. On the patient side of the absorbent layer 105, an adhesive 135 surrounds each of the femoral access regions 115. In the new, sterile condition, the femoral access regions 115 each have plain liners 140 attached to the patient side of the surgical drape by the adhesive 135. In the cross-sectional view, the thicknesses of the absorbent layer 105 and the impervious layer 110 are enlarged for clarity, but the layers are only a few mils in thickness, thus it is possible for the plain liners 140 to be adhered to the surgical drape by the adhesive 135. When the surgical drape is to be used, the appropriate plain liner 140 is removed in order to expose the adhesive 135 so that the surgical drape may be adhered to the patient at the location of the patient's body where access to the patient is desired.

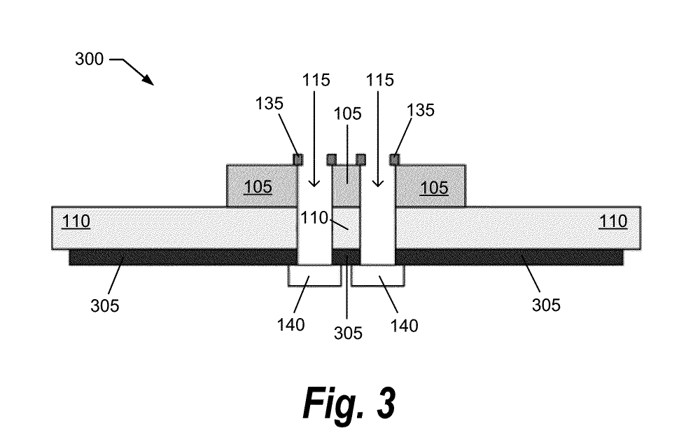

FIG. 3 is a cross-sectional view along the femoral access regions of a surgical drape with femoral and pedal access, in accordance with some embodiments.

In some embodiments in a cross-sectional view along the femoral access regions of a surgical drape with femoral and pedal access, similar to FIG. 2, the surgical drape 300 comprises a substantially full length absorbent layer 105 attached to one side of a substantially rectangular, full length, impervious layer 110 and a third layer 305 attached to the impervious layer 110 opposite the absorbent layer 105 with femoral access regions 115 and pedal access regions. The femoral access regions 115 and pedal access regions extend through the absorbent layer 105, the impervious layer 110, and the third layer 305. The absorbent layer 105 is designed to manage the fluids that exist during a surgical procedure and the impervious layer 110 reduces the movement of these fluids to unwanted areas. The third layer 305 is used to provide for a comfortable material to be in contact with the patient.

In some embodiments, the femoral access regions 115 comprise two substantially circular fenestrations into the absorbent layer 105 which permit access to the groin area of a patient. On the patient side of the absorbent layer 105, an adhesive 135 surrounds each of the femoral access regions 115. In the new, sterile condition, the femoral access regions 115 each have plain liners 140 attached to the patient side of the surgical drape by the adhesive 135. In the cross-sectional view, the thicknesses of the absorbent layer 105 and the impervious layer 110 are enlarged for clarity, but the layers are only a few mils in thickness, thus it is possible for the plain liners 140 to be adhered to the surgical drape by the adhesive 135. When the surgical drape is to be used, the appropriate plain liner 140 is removed in order to expose the adhesive 135 so that the surgical drape may be adhered to the patient at the location of the patient's body where access to the patient is desired.

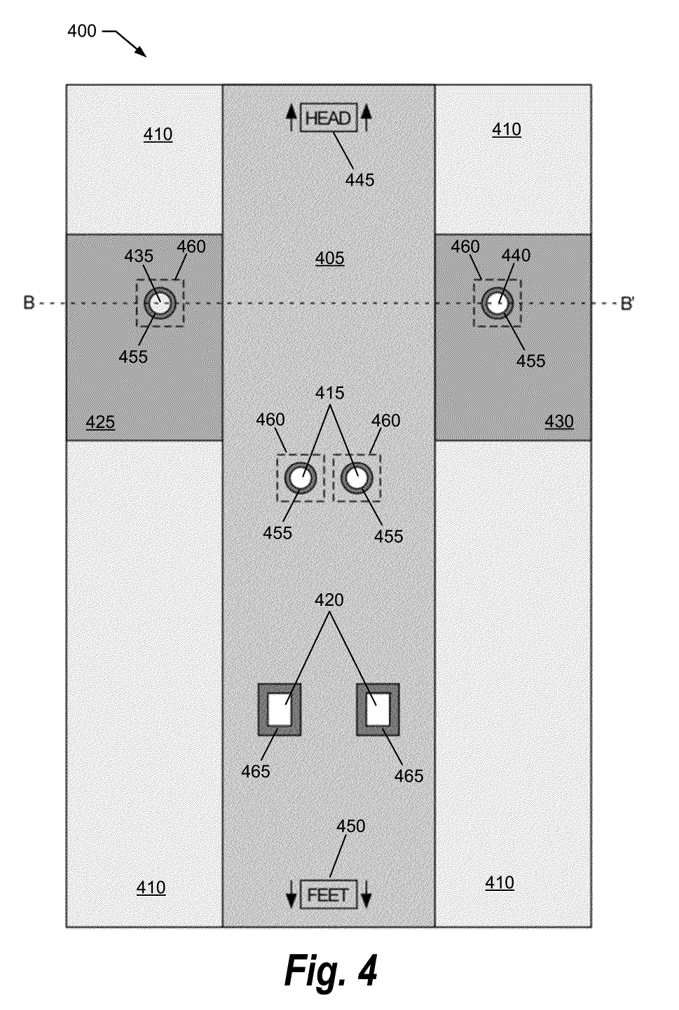

FIG. 4 is a top plan view of a surgical drape with femoral, pedal, and radial access, in accordance with some embodiments.

In some embodiments, the surgical drape 400 comprises a substantially full length absorbent layer 405 attached to a substantially rectangular, full length, impervious layer 410 with femoral access regions 415 and pedal access regions 420 and a second absorbent layer 425 and a third absorbent layer 430 attached to the substantially rectangular, full length, impervious layer 410, each having radial access regions 435 and 440, respectively. In some embodiments, a head stamp 445 is indicated on the absorbent layer 405 along the edge of the absorbent layer 405 on the end closest to the radial access regions 435, 440 and a foot stamp 450 is indicated on the absorbent layer 405 along the edge of the absorbent layer 405 on the end closest to the pedal access regions 420. The femoral access regions 415 and pedal access regions 420 extend through both the absorbent layer 405 and the impervious layer 410. The radial access regions 435 and 440 extend through both the impervious layer 410 and the second absorbent layer 425 and the third absorbent layer 430, respectively. The absorbent layer 405 is designed to manage the fluids that exist during a surgical procedure and the impervious layer 410 reduces the movement of these fluids to unwanted areas.

In some embodiments, the radial access regions 435 and 440 comprise two substantially circular fenestrations into the absorbent layers 425 and 430, respectively, which permit access to the arm/wrist area of a patient. In some embodiments, the radial access regions 435 and 440 comprise circular fenestrations of approximately 5 inches. On the patient side of the absorbent layer 425, 430 an adhesive 455 surrounds each of the radial access regions 435, 440. In the new sterile condition, the radial access regions 435, 440 each have plain liners 460 attached to the surgical drape by the adhesive 455. Since the plain liners 460 are attached on the patient side of the drape, their position is shown as dashed lines, indicating that part of the plain liners 460 are hidden from view when the drape is viewed in a top plan view. When the surgical drape is to be used, the appropriate plain liner 460 is removed in order to expose the adhesive 455 so that the surgical drape may be adhered to the patient at the location of the patient's body where access to the patient is desired.

In some embodiments, the femoral access regions 415 comprise two substantially circular fenestrations into the absorbent layer 405 which permit access to the groin area of a patient. In some embodiments, the femoral access regions 415 comprise circular fenestrations of approximately 5 inches. On the patient side of the absorbent layer 405, an adhesive 455 surrounds each of the femoral access regions 415. In the new, sterile condition, the femoral access regions 415 each have plain liners 460 attached to the patient side of the surgical drape by the adhesive 455. Since the plain liners 460 are attached on the patient side of the drape, their position is shown as dashed lines, indicating that part of the plain liners 460 are hidden from view when the drape is viewed in a top plan view. When the surgical drape is to be used, the appropriate plain liner 460 is removed in order to expose the adhesive 455 so that the surgical drape may be adhered to the patient at the location of the patient's body where access to the patient is desired.

In some embodiments, the pedal access regions 420 comprise two substantially rectangular fenestrations into the absorbent layer 405 which permit access to the ankle/foot area of the patient and a double sided adhesive 465 surrounds each of the pedal access regions 420. In some embodiments, the pedal access regions 420 comprise substantially rectangular fenestrations of approximately 4 inches by 5 inches. When the surgical drape is to be used, the appropriate double sided adhesive 465 is exposed so that the surgical drape may be adhered to the patient at the location of the patient's body where access to the patient is desired.

In some embodiments, a third layer is attached to the impervious layer 410 on the side opposite the absorbent layers 405, 425, 430. In such an embodiment, the radial access regions 435, 440, the femoral access regions 415, and the pedal access regions 420 also extend through the third layer. The third layer is used to provide for a comfortable material to be in contact with the patient. The third layer may comprise a woven or nonwoven fabric layer and/or a layer of fibers.

In some embodiments, the absorbent layer 405, the second absorbent layer 425, and the third absorbent layer 430 comprises a woven or nonwoven fabric layer and/or a layer of fibers. The fabric/fibers may comprise a polymeric film-forming material, such as polypropylene, an absorbent material of Airtex material, Angio material, and/or SMS (“Spunbond+Meltblown+Spunbond Nonwovens”) fabric. The absorbent layer has an average layer thickness of less than about 10 mils. In some embodiments, the absorbent layer 405, the second absorbent layer 425, and the third absorbent layer 430 are all the same material.

In some embodiments, the impervious layer 410 comprises a polymeric film-forming material, such as polyethylene, and has an approximate length of 135 inches, an approximate width of 84 inches, and has an average layer thickness of less than about 2 mils.

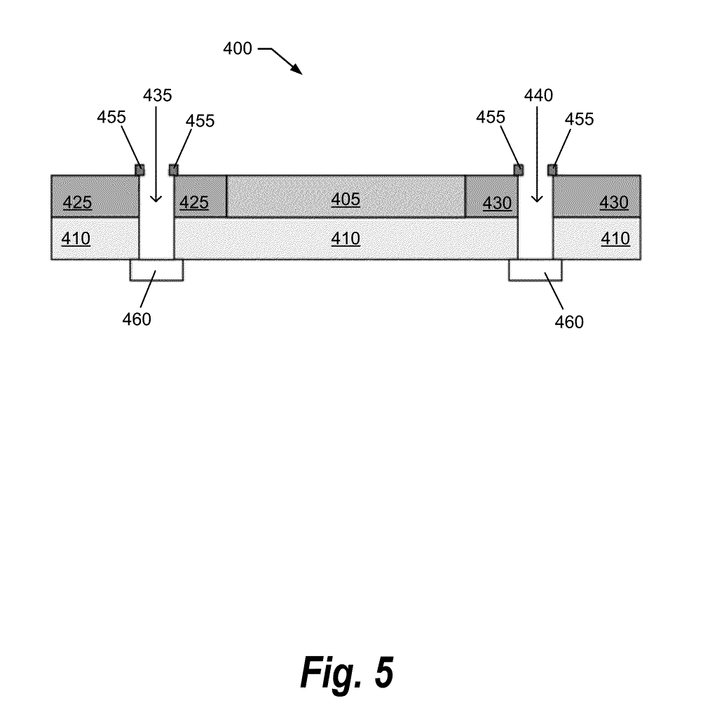

FIG. 5 is a cross-sectional view of the surgical drape of FIG. 4 taken along the line B-B′, in accordance with some embodiments.

In some embodiments, the surgical drape 400 comprises a substantially full length absorbent layer 405 attached to a substantially rectangular, full length, impervious layer 410 with femoral access regions and pedal access regions and a second absorbent layer 425 and a third absorbent layer 430 attached to the substantially rectangular, full length, impervious layer 410, each having radial access regions 435 and 440, respectively. The femoral access regions and pedal access regions extend through both the absorbent layer 405 and the impervious layer 410. The radial access regions 435 and 440 extend through both the impervious layer 410 and the second absorbent layer 425 and the third absorbent layer 430, respectively. The absorbent layer 405 is designed to manage the fluids that exist during a surgical procedure and the impervious layer 410 reduces the movement of these fluids to unwanted areas.

In some embodiments, the radial access regions 435 and 440 comprise two substantially circular fenestrations into the absorbent layers 425 and 430, respectively, which permit access to the arm/wrist area of a patient. On the patient side of the absorbent layers 425 and 430, an adhesive 455 surrounds each of the radial access regions 435 and 440. In the new, sterile condition, the radial access regions 435 and 440 each have plain liners 460 attached to the patient side of the surgical drape by the adhesive 455. In the cross-sectional view, the thicknesses of the absorbent layers 405, 425, 430 and the impervious layer 410 are enlarged for clarity, but the layers are only a few mils in thickness, thus it is possible for the plain liners 460 to be adhered to the surgical drape by the adhesive 455. When the surgical drape is to be used, the appropriate plain liner 460 is removed in order to expose the adhesive 455 so that the surgical drape may be adhered to the patient at the location of the patient's body where access to the patient is desired.

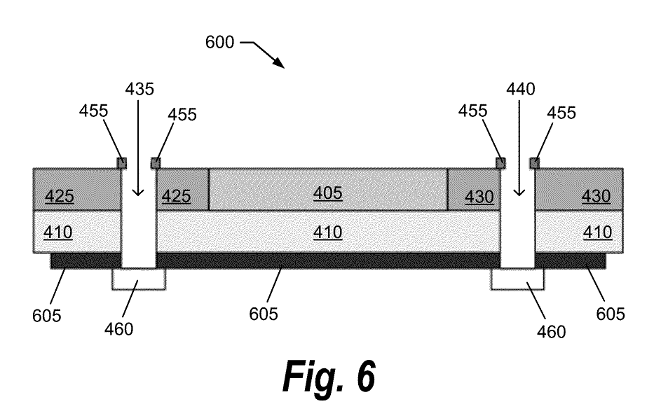

FIG. 6 is a cross-sectional view along the radial access regions of a surgical drape with femoral, pedal, and radial access, in accordance with some embodiments.

In some embodiments in a cross-sectional view along the radial access regions of a surgical drape with femoral, pedal, and radial access, similar to FIG. 5, the surgical drape 600 comprises a substantially full length absorbent layer 405, a second absorbent layer 425, and a third absorbent layer 430 attached to one side of a substantially rectangular, full length, impervious layer 410 and a third layer 605 attached to the impervious layer 410 opposite the absorbent layer 405 with radial access regions 435 and 440 and pedal access regions, the second absorbent layer 425, and the third absorbent layer 430. The femoral access regions, pedal access regions, and radial access regions 435, 440 extend through the absorbent layers 405, 425, 430, the impervious layer 410, and the third layer 605. The absorbent layers 405, 425, 430 are designed to manage the fluids that exist during a surgical procedure and the impervious layer 410 reduces the movement of these fluids to unwanted areas. The third layer 605 is used to provide for a comfortable material to be in contact with the patient.

In some embodiments, the radial access regions 435 and 440 comprise two substantially circular fenestrations into the absorbent layers 425 and 430, respectively, which permit access to the arm/wrist area of a patient. On the patient side of the absorbent layers 425 and 430, an adhesive 455 surrounds each of the radial access regions 435 and 440. In the new, sterile condition, the radial access regions 435 and 440 each have plain liners 460 attached to the patient side of the surgical drape by the adhesive 455. In the cross-sectional view, the thicknesses of the absorbent layers 405, 425, 430, the impervious layer 410, and the third layer 605 are enlarged for clarity, but the layers are only a few mils in thickness, thus it is possible for the plain liners 460 to be adhered to the surgical drape by the adhesive 455. When the surgical drape is to be used, the appropriate plain liner 460 is removed in order to expose the adhesive 455 so that the surgical drape may be adhered to the patient at the location of the patient's body where access to the patient is desired.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

The benefits and advantages that may be provided by the present invention have been described above with regard to specific embodiments. These benefits and advantages, and any elements or limitations that may cause them to occur or to become more pronounced are not to be construed as critical, required, or essential features of any or all of the claims. As used herein, the terms “comprises,” “comprising,” or any other variations thereof, are intended to be interpreted as non-exclusively including the elements or limitations which follow those terms. Accordingly, a system, method, or other embodiment that comprises a set of elements is not limited to only those elements, and may include other elements not expressly listed or inherent to the claimed embodiment.

While the present invention has been described with reference to particular embodiments, it should be understood that the embodiments are illustrative and that the scope of the invention is not limited to these embodiments. Many variations, modifications, additions, and improvements to the embodiments described above are possible. It is contemplated that these variations, modifications, additions, and improvements fall within the scope of the invention as detailed within the following claims.

1. A surgical drape for interventional cardiology, the surgical drape comprising:
a substantially rectangular impervious layer, wherein the substantially rectangular impervious layer is configured to be draped over a patient;
a first absorbent layer attached on one side of the substantially rectangular impervious layer, wherein the first absorbent layer covers substantially the length and partially the width of the substantially rectangular impervious layer;
one or more pedal access regions through the substantially rectangular impervious layer and the first absorbent layer; and
one or more femoral access regions through the substantially rectangular impervious layer and the first absorbent layer.

2. The surgical drape of claim 1, further comprising:
a second absorbent layer attached on the same side of the substantially rectangular impervious layer as the first absorbent layer;
a third absorbent layer attached on the same side of the substantially rectangular impervious layer as the first absorbent layer and the second absorbent layer;
wherein the second absorbent layer and the third absorbent layer are adjacent to and on opposite the sides of the first absorbent layer; and
one or more radial access regions through the substantially rectangular impervious layer and the second absorbent layer and/or the third absorbent layer.

3. The surgical drape of claim 1, further comprising a patient side layer attached to the side of the substantially rectangular impervious layer opposite the side where the first absorbent layer is attached, wherein the one or more pedal access regions pass through the patient side layer and wherein the one or more femoral access regions pass through the patient side layer.

4. The surgical drape of claim 2, further comprising a patient side layer attached to the side of the substantially rectangular impervious layer opposite the side where the first absorbent layer is attached, wherein the one or more pedal access regions pass through the patient side layer, wherein the one or more femoral access regions pass through the patient side layer, and wherein the one or more radial access regions pass through the patient side layer.

5. The surgical drape of claim 1, further comprising a substantially circular adhesive ring attached to the side of the first absorbent layer opposite the side of the substantially rectangular impervious layer and substantially centered on one of the one or more femoral access regions.

6. The surgical drape of claim 5, further comprising a plain liner adjacent to the side of the substantially rectangular impervious layer opposite the substantially circular adhesive ring, wherein the substantially circular adhesive ring is configured to removably hold the plain liner in place and wherein when the plain liner is removed from the surgical drape, the substantially circular adhesive ring is configured to adhere to the patient.

7. The surgical drape of claim 1, further comprising double sided adhesive adjacent to the one or more pedal access regions, wherein the double sided adhesive is configured to adhere to the patient when the adhesive is exposed.

8. The surgical drape of claim 2, further comprising a second substantially circular adhesive ring attached to the side of the second absorbent layer opposite the side of the substantially rectangular impervious layer and substantially centered on one of the one or more radial access regions.

9. The surgical drape of claim 8, further comprising a second plain liner adjacent to the side of the substantially rectangular impervious layer opposite the second substantially circular adhesive ring, wherein the second substantially circular adhesive ring is configured to removably hold the second plain liner in place and wherein when the second plain liner is removed from the surgical drape, the second substantially circular adhesive ring is configured to adhere to the patient.

10. The surgical drape of claim 2, further comprising a third substantially circular adhesive ring attached to the side of the third absorbent layer opposite the side of the substantially rectangular impervious layer and substantially centered on one of the one or more radial access regions.

11. The surgical drape of claim 10, further comprising a third plain liner adjacent to the side of the substantially rectangular impervious layer opposite the third substantially circular adhesive ring, wherein the third substantially circular adhesive ring is configured to removably hold the third plain liner in place and wherein when the third plain liner is removed from the surgical drape, the third substantially circular adhesive ring is configured to adhere to the patient.

12. The surgical drape of claim 2, wherein the first absorbent layer, the second absorbent layer, and the third absorbent layer compromise the same material.

13. The surgical drape of claim 1, further comprising a first stamp configured to indicate placement of the surgical drape relative to the patient, wherein the first stamp points in the direction of the head of the patient.

14. The surgical drape of claim 1, further comprising a second stamp configured to indicate placement of the surgical drape relative to the patient, wherein the second stamp points in the direction of the feet of the patient.
